(19) United States
(12) Patent Application Publication
(10) Pub. No.: US 2006/0084166 A1
(43) Pub. Date: Apr. 20, 2006

(54) Compost tea delivery system and method

(76) Inventor: Jennifer Appel, Houston, TX (US)

Correspondence Address: PATTERSON & SHERIDAN, L.L.P., 3040 POST OAK BOULEVARD, SUITE 1500, HOUSTON, TX, 77056, US

(21) Appl. No.: 11/252,396

(22) Filed: Oct. 18, 2005

Related U.S. Application Data
(60) Provisional application No. 60/619,625, filed on Oct. 18, 2004.

(51) Int. Cl. C05F 17/02; C12M 1/36

(52) U.S. Cl. 435/286.5; 435/290.4

(57) ABSTRACT

The present invention is generally directed to a method and a system for producing and delivering compost tea to a predetermined location. In one aspect, a compost tea delivery system is provided. The system includes a compost tea production unit configured to produce a compost tea and a compost tea tank for storing the compost tea. The compost tea tank is interconnected with the compost tea production unit via a conduit system. The system also includes a control member configured to control the flow of the compost tea through the conduit system as the compost tea is delivered to a predetermined location. In another aspect, a system for delivering compost tea is provided. In yet another aspect, a method of delivering compost tea to a predetermined location is provided.

### CROSS-REFERENCE TO RELATED APPLICATIONS

This application claims benefit of U.S. provisional patent application Ser. No. 60/619,625, filed Oct. 18, 2004, which is herein incorporated by reference.

### BACKGROUND OF THE INVENTION

1. Field of the Invention

The present invention generally relates to compost teas. More particularly, the invention relates to the production and the delivery of compost teas.

2. Description of the Related Art

Compost teas are becoming recognized as a beneficial supply of organic nutrients and microorganisms for the feeding and enhancement of plants, trees, grass, and other growing plant life (herein broadly termed “flora”). The “tea” is a derivative aqueous extract of compost exposed to water that produces nutrients and microorganisms. The compost tea is spread on the flora to increase growth, control disease, and enhance the media in which the flora is grown.

Prior efforts appear to have concentrated on improvements relating to the production of the compost tea. An early production method was to simply mix water and compost, ferment for a time period, and then separate the liquid from particulate material and apply it to crops and soil. As technology increased, it was determined that a key component to the production of compost tea is to supply sufficient oxygen to the compost/liquid mixture in order to rapidly develop high quality compost tea. The oxygen can be added to the compost/liquid in numerous ways. For instance, a pump in a bioreactor may be used to circulate the compost/liquid in a tank thereby adding oxygen from surrounding air as described in U.S. Pat. No. 6,168,949, which is herein incorporated by reference. In another example, air is pumped into membrane disk diffuser modules in a tank and the liquid is sparged with fine bubbles to thoroughly agitate the liquid, extract nutrients and microorganisms from the compost, and to ensure a high concentration of dissolved oxygen in the liquid as described in U.S. Pat. No. 6,649,405, which is herein incorporated by reference. However, this technology does not address the need to integrate the production of the compost tea into a delivery system that is practical. The existence of the compost teas production does little to the field if it is not easily and practically deliverable to the site for use.

Thus, there remains a need for a delivery system capable of delivering the compost tea to the site for use.

### SUMMARY OF THE INVENTION

The present invention is generally directed to a method and a system for producing and delivering compost tea to a predetermined location. In one aspect, a compost tea delivery system is provided. The system includes a compost tea production unit configured to produce a compost tea and a compost tea tank for storing the compost tea. The compost tea tank is interconnected with the compost tea production unit via a conduit system. The system also includes a control member configured to control the flow of the compost tea through the conduit system as the compost tea is delivered to a predetermined location.

In another aspect, a system for delivering compost tea is provided. The system includes a compost tea production unit configured to produce a compost tea and a compost tea tank for storing the compost tea. The system further includes a conduit and valve arrangement for interconnecting the compost tea production unit to the compost tea tank and for delivering the compost tea to a predetermined location. Additionally, the system includes a control member for selectively controlling the conduit and valve arrangement.

In yet another aspect, a method of delivering compost tea to a predetermined location is provided. The method includes producing a first portion of compost tea in a compost tea production unit and moving the first portion of compost tea from the compost tea production unit to a compost tea tank. The method further includes controlling the delivery of the first portion of compost tea to the predetermined location by a controller member while a second portion of compost tea is produced in the compost tea production unit.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a view illustrating a compost tea delivery system.

FIG. 2 is a view illustrating another compost tea delivery system.

### DETAILED DESCRIPTION

The present invention is generally directed to a method and a system for producing and delivering compost tea to a predetermined location. Various terms as used herein are defined below. To the extent a term used in a claim is not defined below, it should be given the broadest definition persons in the pertinent art have given that term, as reflected in printed publications and issued patents. In the description that follows, like parts are marked throughout the specification and drawings with the same number indicator. The drawings may be, but are not necessarily, to scale, and the proportions of certain parts have been exaggerated to better illustrate details and features of the invention.

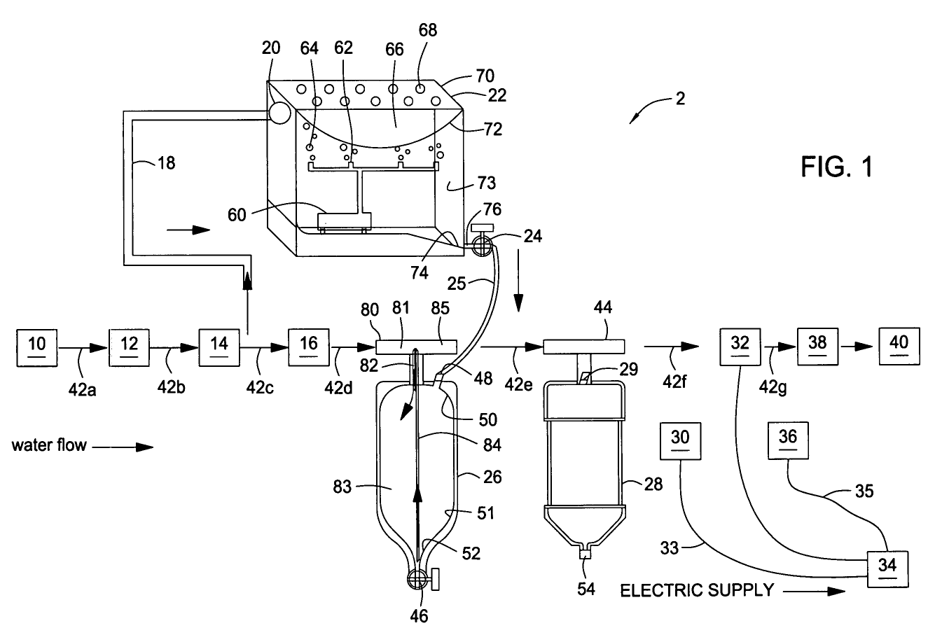

FIG. 1 is a view illustrating one embodiment of a compost tea delivery system 2. Generally, the compost tea delivery system 2 is used to produce a compost tea and selectively deliver the compost tea to a predetermined location, such as the flora 40. As will be described herein, the delivery system 2 includes a compost tea production unit 22 for producing the compost tea, a compost tea tank 26 for storing the compost tea, various valves, conduits, sensors, a controller, and an irrigation system to provide an efficient and a practical delivery mechanism of the compost tea to the predetermined location.

As shown in FIG. 1, a water source 10 is coupled to a master valve 12. The water source 10 can be from a gravity feed or a pumped pressurized source and can originate from ground water, surface water, rainwater, or other water sources. The term “water” is used broadly and generally includes a fluid having at least a portion of its composition as water.

The water source 10 is coupled to the master valve 12 via a conduit 42a. For ease of explanation, the miscellaneous conduits coupling the various components are generally labeled with the number 42 as the primary element number. The conduits can be tubing, pipes, or other liquid conducting components. The term “couple,” coupled,” “coupling,” and like terms are used broadly herein and can include any method or device for communicating, securing, attaching, or otherwise associating, for example, mechanically, fluidicly, magnetically, electrically, chemically, directly or indirectly with intermediate elements, one or more pieces of members together and can further include integrally forming one functional member with another.

As illustrated in FIG. 1, the compost tea production unit 22 is shown and described as a brewer machine. It should be understood, however, that the compost tea production unit 22 may any type of compost tea production system, without departing from principles of the present invention. For instance, the compost tea production unit 22 could be an extractor, wherein a high pressure water jet forces water through the compost or vitamins to remove the beneficial organisms to produce the compost tea.

The master valve 12 provides an overall shut-off to the system for maintenance and other functions. In general, the valves described in reference to FIG. 1 are manually operated which can provide a more cost-effective system. Optionally, the master valve 12 may be coupled to a backflow preventer 14. The backflow preventer 14 may be any device capable of preventing unintended backflow into a main water system. Some examples of backflow preventers 14 are atmospheric vacuum breakers, pressure vacuum breakers, double check valves, and reduced pressure principle vacuum breakers.

The backflow preventer 14 may be coupled to another valve 16 that is configured to selectively segregate portions of the system. For example, the valve 16 can be closed when a compost tea tank 26 or a compost tea production unit 22 is filling and the valve 16 can be opened for irrigation and feeding operations. In another example, the valve 16 can be closed when a fertigation unit 28 is filling.

As illustrated in FIG. 1, a conduit 18 can be coupled to the conduit 42c to provide water to the portion of the system 2 used to produce the compost tea. A valve 20 can be coupled to the conduit 18 to control inlet of water to the unit 22. During operation of the system 2, the valve 20 is typically closed except when filling the compost tea production unit 22.

To expedite the generation of the compost tea in the compost tea production unit 22, oxygen is typically pumped into the liquid. In one embodiment, the compost tea production unit 22 includes a pump 60 that draws air or other gases containing oxygen from the environment surrounding the system 22 or from a storage tank (not shown), and then the pump 60 urges the gas through a manifold 62 and into the liquid in the unit 22. As a plurality of gas bubbles 64 move through the liquid, the gas bubbles 64 enrich the oxygen content in the liquid, thereby promoting microorganism growth. After moving through the liquid, the bubbles 64 permeate through a compost area 66 holding a quantity of compost. To reduce an over-pressurized condition in the unit 22, one or more openings 68 may be formed in a removable lid 70 on the unit 22 or another portion of the unit 22 to allow the bubbles 64 to escape. The bubbles 64 may also be used to create passive circulation within the compost tea to further increase the efficiency of production. As shown in FIG. 1, a removable mesh screen 72 is configured to retain the compost in a localized area and thereby substantially reduces or eliminates the amount of compost in the liquid that exits the unit 22.

After a predetermined time, the liquid portion of the compost tea is drained from the unit 22 by passing the compost tea through a screen 73 and subsequently exiting the unit 22 through an outlet 76. A sloped surface 74, as described in U.S. Publication US20050160670 which is incorporated herein by reference, may be used to increase the amount of compost tea drained from the unit 22. A valve 24 may be attached to the outlet 76 to control the outflow of the liquid portion of the compost tea from the unit 22. In one embodiment, the compost tea production unit 22 is sized approximately with the same capacity as the compost tea tank 26 in order to assist is providing fresh supplies of compost tea for each cycle. Additionally, the compost may be manually removed from the compost tea production unit 22.

As illustrated, the compost tea production unit 22 is coupled to the compost tea tank 26 via a conduit 25. In one embodiment, the conduit 25 can be a flexible hose that can be readily removed for cleaning or replacement. At a predetermined time the valve 24 and a valve 48 may be opened to allow fluid communication between the unit 22 and the tank 26. The valves 24, 48 allow the system's pressurized portions to be isolated from the conduit 25 while the production unit 22 is operating and while the water is pressurizing the conduits and other components.

In another embodiment, either valve 24 or valve 48 may be eliminated since the valve 24, 48 has overlapping functions. The compost tea tank 26 receives the compost tea from the compost tea production unit 22 and subsequently dispenses the tea dependent on the water flow through the tea tank 26, for example, through a proportioning valve. Thus, the production unit 22 can be freed to produce more compost tea, while the previously produced compost tea is being systematically distributed into the delivery system and ultimately to the predetermined location. In other words, the compost tea production unit 22 and the compost tea tank 26 work together to provide a substantially continuous flow of compost tea to the predetermined location. For instance, as the compost tea production unit 22 produces a batch of compost tea, the compost tea tank 26 distributes the previously made batch of tea.

The compost tea tank 26 includes a mixing conduit 80 having an entry zone 81 and an exit zone 85 downstream of the entry zone 81. In general, water from the conduit 42d enters the mixing conduit 80 and flows into the entry zone 81. The mixing conduit 80 can use a proportioning mixer, such as, and without limitation, disclosed in U.S. Pat. No. 4,846,214, which is incorporated herein by reference. At least a portion of the water in the entry zone 81 flows through an opening 82 formed in the compost tea tank 26 and enters the main cavity 83 of the tea tank 26. As water enters the main cavity 83, the compost tea currently in the main cavity is pushed downward to a lower portion of the tank 26. A conduit 84, having an inlet in the tank 26 and generally near the bottom of the tank, allows the compost tea to flow therethrough. As a result, the compost tea flows up conduit 84 and mixes with the water from the entry zone 81 and flows into the exit zone 85 and out the compost tea tank 26. As illustrated, a valve 46 can be used to drain the compost tea tank 26. The compost tea tank 26 can beneficially be drained regularly to avoid creating stagnant liquid and disrupting the microorganisms and other beneficial characteristics of the fluid in the tea tank 26.

Advantageously, the compost tea tank 26 can be formed with little or no lips, joints, seams, or other protrusions. Further, the compost tea tank 26 is advantageously formed with radii 50, 51, 52 for the interfaces between the wall sides, top, and bottom of the tea tank 26. It has been discovered that protrusions or abrupt changes in a flow path in a tank holding compost tea can create biofilms. These biofilms potentially stagnate portions of the compost tea and generate anaerobic bacteria, which can be deadly to the aerobic microorganisms in the compost tea. The compost tea can rapidly become less than advantageous and potentially poisonous to the flora 40 and site upon which it is delivered. Advantageously, the compost tea tank 26 is formed as a single piece, such as in a single mold, to reduce the joints and seams upon which the biofilms can occur. Further, the compost tea tank 26 generally has sufficiently thick walls to withstand the pressurized liquid inside the tank.

As illustrated in FIG. 1, the compost tea tank 26 can be coupled to a fertigation unit 28. The fertigation unit 28 provides a means to include liquid fertilizers and other dissolvable additives into the system 2 to further enhance the liquid delivered to the predetermined location, such as the flora 40. The fertigation unit 28 typically includes a fill cap 29 through which the materials are added to the fertigation unit 28. Upstream liquid can enter the unit 28 through a mixing conduit 44, flow into the main body of the fertigation unit 28, pressure the enhanced liquid out of the main body, and into the mixing conduit 44 to be mixed with the liquid flowing therethrough. A valve 54 can be used to drain or flush the tank.

The fertigation unit 28 can be coupled to one or more zone valves 32 for delivering the liquid to the site's flora 40, if the site has multiple zones. The zone valves 32 can be controlled with a controller 34.

The controller 34 can control various parameters such as days, duration, start/stop times, manual mode, test mode, and other programming features for multiple zones as desired. Default settings can be provided, such as run time maximum and a water increase/decrease budget of 0% to about 200%. Further the controller 34 can have a “feed” cycle designed to operate for a set operation, including time, days, and duration of a sequence to provide the compost tea. Such a cycle could be set independently of an irrigating cycle.

Further, one or more sensors 30 can be coupled to the system 2 and provide input to the controller 34. For example, one or more sensors 30 can include temperature useful for detecting freeze conditions, high heat conditions, wind, rain, and other sensed parameters. The sensors 30 can be coupled to the controller 34 through a line 33, through wireless communications, or otherwise. In some embodiments, the sensors 30 can be directly coupled to the zone valves 32 and override a signal from the controller 34. The sensors 30 provide input for controlling the timing, duration, activation, and other parameters of the system 2. In other words, the sensors 30 provide input that affects the overall operation of the zone valves 32, regardless of the zone.

One or more other sensors 36 can be used to affect the operation of specific zones. For example, and without limitation, one or more sensors 36 can provide moisture input per zone or per subgroup of zones to the controller 34 through line 33, through wireless communications, or otherwise. The input can adjust the operation of the particular zone or subgroup based on the amount of moisture in the ground, plant, or tree. Other types of input per zone or subgroup of zones can be used.

The zone valves 32 can be coupled to an outlet of the liquid from the delivery system 2 to the site. For example and without limitation, the outlet can include one or more irrigation heads 38. The irrigation heads 38 can dispense the liquid to the flora 40.

In operation, the valves are configured to allow water to pass through the different valves, compost tea tank 26, fertigation unit (if present), and zone valves. The compost tea production unit 22 can produce the tea and periodically release the compost tea to the compost tea tank 26 for mixing with the water passing therethrough. One example of a sequence is described below. To operate the irrigation cycle, the valve 16 is opened and the valves 20, 24, 46, and 48 are generally closed. To operate a compost tea production cycle, the valves 16, 24, 48 are closed, the valve 20 is opened to allow water to fill to a desired level in the compost tea production unit 22, and then valve 20 is closed. At the same time, the pump 60 operates for a specified time to pump gases into the compost tea production unit 22. The valve 16 can be open to allow irrigation to occur while the compost tea production unit 22 is operating. To empty the tea tank 26, the valve 16 is closed and the valves 46, 48 are opened. After the tea tank 26 is emptied, valve 46 is closed and valve 24 is opened. After the tea tank 26 is filled, valves 24, 48 are closed and valve 16 is opened. Water flowing through the delivery system is mixed with the compost tea and is delivered to the site. The controller 34 can have an independent “feed” cycle set at different times, dates, durations, and so forth than a regular irrigation cycle to properly dispense the compost tea to the various portions of the site.

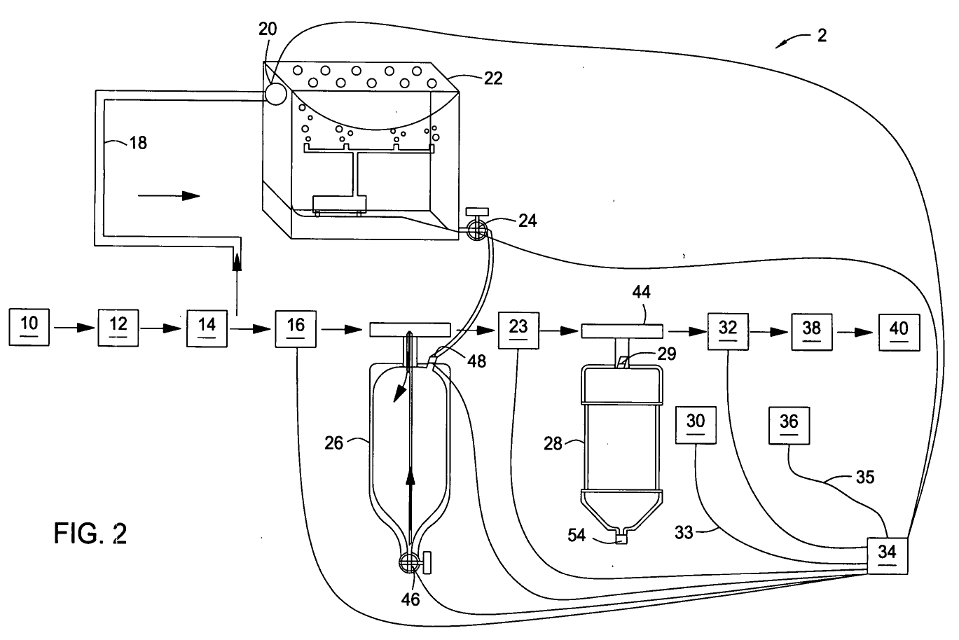

FIG. 2 is a schematic diagram of a partially automated delivery system 3. As illustrated, the system 3 has an overall design similar to the system 2 as illustrated in FIG. 1 with the additional capability of the automation of various valves of the system 3 through a controller. For convenience, the components in the system 3 that are similar to the components in the system 2 will be labeled with the same number indicator. In general, a water source 10 provides water to the system 3 through a valve 12 and a backflow preventer 14, if used. A conduit 18 branches from the primary conduit path fluidicly coupling the various components handling the water and compost tea and mixtures thereof. The conduit 18 provides water to the compost tea production unit 22 through a valve 20. An outlet valve 24 coupled to the compost tea production unit 22 allows the compost tea produced therein to flow to the compost tea tank 26, where the tea is mixed with the water source. An intermediate valve 48 coupled to an inlet of the compost tea tank 26 can control the entry of the fluid into the compost tea tank 26. A valve 46 can be coupled to a drain portion of the compost tea tank 26 at the bottom. A fertigation unit 28 can be included in the system 3, such as coupled to the compost tea tank 26. A valve 23 can be disposed upstream of the fertigation unit 28 to control the fluid entering the fertigation unit. The valve 23 can be closed when supplying fertilizer or other beneficial products into the fertigation unit and open when allowing the fluid to flow therethrough to mix the products with the fluid. The fertigation unit 28 can be coupled to one or more zone valves 32. The zone valves 32 can dispense the compost tea fluid to one or more zones of the site to which the compost tea is delivered. One or more overall sensors 30 and one or more zone or subgroup specific sensors 36 can provide input for control of the system 3.

A controller 34 can be used to control the system 3 through the operation of the valves. The controller 34 can provide timing, duration, manual and test modes, and so forth for irrigation. The controller 34 can also provide for the control of the compost tea production. The controller 34 can be coupled either through wires or wirelessly to various valves in the system 3. For example, the system 3 can irrigate while the compost tea production unit 22 is producing compost tea, and then provide a feed cycle separate from the irrigation cycle or in conjunction therewith. The controller can be provided with a manual override on the automatic valves for cleaning, maintenance, and other operations.

By way of example and without limitation to other sequences, the controller 34 can control various valves for irrigation in the following manner. To operate the irrigation cycle, the valves 16, 23 are open, and the valves 20, 24, 46, and 48 are generally closed. To operate a compost tea production cycle, the valves 16, 24, 48 are closed, and the valve 20 is open to allow water to fill to a desired level in the compost tea production unit 22 and valve 20 is closed. The pump 60 operates for a specified period to pump gases into the compost tea production unit 22. The valves 16, 23 can be open to allow irrigation to occur while the compost tea production unit 22 is operating. To empty the tea tank 26, generally after the compost tea is ready in the production unit 22, valves 16, 23 are closed and valves 46, 48 are opened. After a time delay when the tea tank 26 is emptied, the valve 46 is closed, and valve 24 is open to fill the tea tank 26. After the tea tank 26 is filled, the valves 24, 48 are closed and the valves 16, 23 are open. Further, the valve 23 can have a manual option to fill the fertigation unit 28 with fertilizer or other products. Water flowing through the delivery system 3 is mixed with the compost tea and is delivered to the site. The controller 34 can have an independent “feed” cycle set at different times, dates, durations, and so forth than a regular irrigation cycle to properly dispense the compost tea to the various portions of the site.

Various basics of the invention have been explained herein. The various techniques and devices disclosed represent a portion of that which those skilled in the art would readily understand from the teachings of this application. Those with ordinary skill in the art can add details for the implementation thereof. Such details may be added to the disclosure in another application based on this application and it is believed that the inclusion of such details does not add new subject matter to the application. The accompanying figures may contain additional information not specifically discussed in the text and such information may be described in a later application without adding new subject matter. Additionally, various combinations and permutations of all elements or applications can be created and presented. All can be done to optimize performance in a specific application.

The various steps described herein can be combined with other steps, can occur in a variety of sequences unless otherwise specifically limited, various steps can be interlineated with the stated steps, and the stated steps can be split into multiple steps. Unless the context requires otherwise, the word “comprise” or variations such as “comprises” or “comprising”, should be understood to imply the inclusion of at least the stated element or step or group of elements or steps or equivalents thereof, and not the exclusion of any other element or step or group of elements or steps or equivalents thereof.

Further, any documents to which reference is made in the application for this patent as well as all references listed in any list of references filed with the application are hereby incorporated by reference. However, to the extent statements might be considered inconsistent with the patenting of this invention such statements are expressly not to be considered as made by the Applicant.

Also, any directions such as “top,” “bottom,” “left,” “right,” “upper,” “lower,” and other directions and orientations are described herein for clarity in reference to the figures and are not to be limiting of the actual device or system or use of the device or system. The device or system may be used in a number of directions and orientations.

While the foregoing is directed to embodiments of the present invention, other and further embodiments of the invention may be devised without departing from the basic scope thereof, and the scope thereof is determined by the claims that follow.

1. A compost tea delivery system comprising:
a compost tea production unit configured to produce a compost tea;
a compost tea tank for storing the compost tea, the compost tea tank is interconnected with the compost tea production unit via a conduit system;
a control member configured to control the flow of the compost tea through the conduit system as the compost tea is delivered to a predetermined location.

2. The system of claim 1, further including a fertigation unit for adding additives into the system.

3. The system of claim 1, further including a backflow preventer disposed between a water supply and the compost tea production unit.

4. The system of claim 3, wherein the backflow preventer is constructed and arranged to prevent unintended backflow from the production unit into the water supply.

5. The system of claim 1, wherein the compost tea production unit includes a pump and a manifold arrangement for injecting a gas into the compost tea.

6. The system of claim 1, wherein the compost tea production unit includes a sloped surface configured to increase the amount of compost tea drained from the compost tea production unit.

7. The system of claim 1, wherein the compost tea tank is constructed and arranged with substantially no protrusions or abrupt changes within a flow path through the compost tea tank.

8. The system of claim 1, further including a valve between the compost tea production unit and the compost tea tank for controlling the flow of the compost tea.

9. The system of claim 1, wherein the compost tea production unit and the compost tea tank have substantially the same holding capacity.

10. The system of claim 1, wherein the control member includes at least one sensor configured to measure at least one environmental parameter at the predetermined location.

11. A system for delivering a compost tea, the system comprising:
a compost tea production unit configured to produce the compost tea;
a compost tea tank for storing the compost tea;
a conduit and valve arrangement for interconnecting the compost tea production unit to the compost tea tank and for delivering the compost tea to a predetermined location; and
a control member for selectively controlling the conduit and valve arrangement.

12. The system of claim 11, wherein the conduit and valve arrangement includes a valve between the compost tea production unit and the compost tea tank, the valve is controlled by the control member.

13. The system of claim 11, wherein the control member includes at least one sensor configured to measure at least one environmental parameter at the predetermined location.

14. The system of claim 11, wherein the conduit and valve arrangement includes at least one irrigation head to distribute the compost tea at the predetermined location.

15. The system of claim 11, wherein the compost tea production unit includes a sloped surface configured to increase the amount of compost tea drained from the compost tea production unit.

16. A method of delivering compost tea to a predetermined location, the method comprising:
producing a first portion of compost tea in a compost tea production unit;
moving the first portion of compost tea from the compost tea production unit to a compost tea tank; and
controlling the delivery of the first portion of compost tea to the predetermined location by a controller member while a second portion of compost tea is produced in the compost tea production unit.

17. The method of claim 16, further including sensing environmental data at the predetermined location.

18. The method of claim 17, further including using the environmental data to control the delivery of the compost tea to the predetermined location.

19. The method of claim 16, further including controlling the flow of the compost tea between the compost tea production unit and the compost tea tank.

20. The method of claim 16, further including injecting an additive into the compost tea prior to delivering the compost tea to the predetermined location.
